(19) United States
(12) Patent Application Publication
(10) Pub. No.: US 2008/0170998 A1
(43) Pub. Date: Jul. 17, 2008

(54) Method and composition for treating or prevending an oral cavity

(75) Inventors: Satoshi Shizukuishi, Osaka (JP); Hideki Nagata, Osaka (JP); Jun Hiraki, Tokyo (JP); Masami Todokoro, Tokyo (JP)

Correspondence Address: HOGAN & HARTSON LLP;IP GROUP, COLUMBIA SQUARE, 555 THIRTEENTH STREET, N.W., WASHINGTON, DC, 20004, US

(73) Assignees: OSAKA UNIVERSITY; CHISSO CORPORATION

(21) Appl. No.: 12/071,770

(22) Filed: Feb. 26, 2008

Related U.S. Application Data
(62) Division of application No. 11/509,815, filed on Aug. 25, 2006.

(30) Foreign Application Priority Data
Aug. 26, 2005 (JP) 2005-246639

(51) Int. Cl. A61K 9/68; A61K 38/02; A61K 38/08; A61K 8/64

(52) U.S. Cl. 424/48; 514/2; 514/17; 424/49

(57) ABSTRACT

The invention relates to a composition for treating or preventing an oral cavity by preventing adhesion of Porphyromonas gingivalis as periodontopathic bacterium to oral tissue. The invention also relates to a composition for treating or preventing an oral cavity containing a peptide in which arginine and histidine bind alternately. Preferably, the invention includes a composition for treating or preventing an oral cavity including the peptide of a pentamer of (arginine-histidine).

### CROSS REFERENCE TO RELATED APPLICATIONS

This application is a divisional application of U.S. patent application Ser. No. 11/509,815 (filed Aug. 25, 2006), which claims priority under 35 U.S.C. § 119 to Japanese Patent Application No. JP 2005-246639, filed Aug. 26, 2005, each of which applications is expressly incorporated herein by reference in its entirety.

### BACKGROUND OF THE INVENTION

1. Field of the Invention

The invention relates to a composition for an oral cavity which has a suppressing effect on the adhesion of oral bacteria to an oral tissue and is used for a prevention and treatment of periodontal diseases.

2. Description of the Related Art

Periodontal diseases are caused by dental plaque, which is an aggregate of bacteria adhering to teeth. In this plaque, the bacteria which seem to deeply involve the periodontal diseases, have been found. Examples of these bacteria include: black-pigmented anaerobic rods such as Porphyromonas gingivalis, Prevotella intermedia, and Tannerella forsythia; Actinobacillus actinomycetemcomitans; and Fusobacterium nucleatum. Those bacteria produce a variety of virulence factor such as an adhesin, capsular polysaccharide, tissue degradative enzyme, organic acid, sulfide, and endotoxin, to thereby cause the periodontal diseases.

In particular, Porphyromonas gingivalis is considered as a significant pathogenic bacterium of the periodintal diseases because Porphyromonas gingivalis produces an arginine-specific protease (Arg-gingipain) or lysine-specific protease (Lys-gingipain) destroying a periodontal tissue, and plural study groups indicate etiologic relevance between this bacterium and adult periodontitis.

Preventing the adhesion of Porphyromonas gingivalis as the significant pathogenic bacterium is considered to be effective in preventing the periodontal diseases.

For a technique to prevent the adhesion of Porphyromonas gingivalis to oral tissue, for example, it has been reported that lysine and arginine inhibit the adhesion of Porphyromonas gingivalis to a buccal mucosa epithelial cell (Journal of Dental Health 38: 590-591, 1988). It has also been reported that a synthetic peptide containing 8 residues to 24 residues of a primary structure of histatin inhibits hemagglutination activity of Porphyromonas gingivalis (Archs Oral Biol. Vol. 35, No. 9, p 775-777 (1990)). It has further been reported that arginine and guanidinated albumin inhibit hemagglutination activity of exohemagglutinin of Porphyromonas gingivalis (Infection and Immunity, Vol. 52, No. 2, p 421-427 (1986)) and there is a report that lysine and arginine also inhibit the hemagglutination activity (Infection and Immunity, Vol. 54, No. 3, p 659-665 (1986)).

On the other hand, there is a report relating to a composition for oral cavity involving the use of a peptide in which two or more basic amino acids successively bind in a molecule that is effective in suppressing the adhesion of Porphyromonas gingivalis to a gingival epithelia cell or saliva-coated hydroxyapatite (JP 07-68111 B).

Each of the foregoing reports, however, is limited in that (a) they exhibit an aggregation inhibitory effect is weak or (b) require an expensive chemical (e.g., a synthesized long chain peptide) to be used.

Further, there is a report that polyamino acid or those derivatives produced by microbial fermentation, each of which is represented by Formula (1), have antimicrobial properties (WO2004/014944 A1).

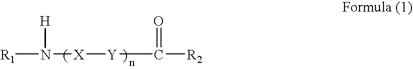

wherein, X represents an arginine residue and the like, and Y represents a histidine residue and the like.

### BRIEF SUMMARY OF THE INVENTION

It is an object of the invention to provide a composition for oral cavity, which prevents an adhesion of Porphyromonas gingivalis being a periodontopathic bacterium to an oral tissue.

It has been observed that a specific peptide produced by a certain microbe by fermentation does not have an antimicrobial activity (a growth inhibitory activity) for Porphyromonas gingivalis, but inhibits hemagglutination activity of Porphyromonas gingivalis, adhesion of Prophyromonas gingivalis to saliva-coated hydroxyapatite, and coaggregation of Porphyromonas gingivalis and Streptococcus oralis. Thus, the invention relates generally to the use of a peptide produced by a certain microbe as a composition for oral cavity prevention and treatment.

The invention includes:

(1) A composition for oral cavity including a peptide in which arginine and histidine bind alternately.

(2) A composition for oral cavity according to (1), wherein the peptide has a structure represented by the following Formula (I) or (II):

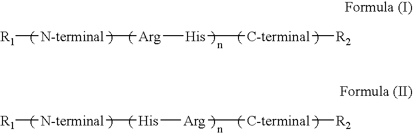

wherein, Arg represents arginine, and H is represents histidine, in addition, R₁ represents hydrogen, sugar, acyl, biotinyl, thiol, phenol, or indole, R₂ represents a hydroxyl group, sugar, acyl, biotinyl, thiol, phenol, or indole, and n represents an integer of 2 or more.

(3) A composition for oral cavity according to (1) or (2), wherein the peptide has a structure represented by the following Formula (1):

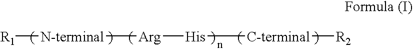

wherein, Arg represents arginine, and H is represents histidine, in addition, R₁ represents hydrogen, sugar, acyl, biotinyl, thiol, phenol, or indole, R₂ represents a hydroxyl group, sugar, acyl, biotinyl, thiol, phenol, or indole, and n represents an integer of 2 or more.

(4) A composition for oral cavity according to any one of (1) to (3), wherein the ratio of D-arginine and L-arginine in the arginine is approximately 10:90 to approximately 0:100.

(5) A composition for oral cavity according to any one of (1) to (4), wherein the arginine is L-arginine.

(6) A composition for oral cavity according to any one of (1) to (5), wherein a ratio of D-histidine and L-histidine in the histidine is approximately 100:0 to approximately 70:30.

(7) A composition for oral cavity according to any one of (1) to (6), wherein the histidine is D-histidine.

(8) A composition for oral cavity according to any one of (1) to (7), wherein the peptide is any one of monomer to icosamer of (arginine-histidine).

(9) A composition for oral cavity according to any one of (1) to (8), wherein the peptide is a pentamer of (arginine-histidine).

(10) A composition for oral cavity according to any one of (1) to (9), wherein the peptide is a peptide produced by microbial fermentation.

(11) A composition for oral cavity according to any one of (1) to (10), wherein the content of the peptide is approximately 0.001 to approximately 10 wt %.

(12) A composition for oral cavity according to any one of (1) to (11), wherein the content of the peptide is approximately 0.01 to approximately 1 wt %.

(13) A composition for oral cavity according to any one of (1) to (12), wherein the composition inhibits a hemagglutination activity of Porphyromonas gingivalis, an adhesion between Porphyromonas gingivalis and saliva-coated hydroxyapatite beads, and a coaggregation of Porphyromonas gingivalis and Streptococcus oralis.

(14) A composition for oral cavity according to any one of (1) to (13) which is used for prevention of periodontal diseases.

(15) A composition for oral cavity according to any one of (1) to (13) which is used for treatment against periodontal diseases.

(16) A use of the composition for oral cavity according to any one of (1) to (14) in production of a preventive for periodontal diseases.

(17) A use of the composition for oral cavity according to any one of (1) to (14) in production of a therapeutic agent for periodontal diseases.

(18) A method of preventing periodontal diseases that includes administering the composition for oral cavity according to any one of (1) to (13).

(19) A method of treating periodontal diseases that includes administering the composition for oral cavity according to any one of (1) to (13).

A composition for oral cavity of the invention can prevent the adhesion of periodontopathic bacteria to oral tissue, so the composition for oral cavity may be used, for example, as a preventive or therapeutic agent for periodontal diseases.

### BRIEF DESCRIPTION OF THE DRAWING

The accompanying drawings, which are included to provide a further understanding of the invention and are incorporated in and constitute a part of this specification, illustrate embodiments of the invention and together with the description serve to explain the principles of the invention. In the drawings:

FIG. 1 is a graph showing an influence of polyarginyl histidine on the growth of Porphyromonas gingivalis ATCC 33277 strain.

FIG. 2 is a graph showing an influence of polyarginyl histidine on the growth of Porphyromonas gingivalis W50 strain.

FIG. 3 is a graph showing an influence of polyarginyl histidine on the growth of Prevotella intermedia ATCC 49046 strain.

FIG. 4 is a graph showing an influence of polyarginyl histidine on the growth of Prevotella nigrescens ATCC 25261 strain.

FIG. 5 is a graph showing an influence of polyarginyl histidine on the growth of Actinobacillus actinomycetemcomitans ATCC 29523 strain.

FIG. 6 is a graph showing an influence of polyarginyl histidine on the growth of Actinobacillus actinomycetemcomitans Y4 strain.

FIG. 7 is a graph showing an influence of polyarginyl histidine on the growth of Fusobacterium nucleatum ATCC 23726 strain.

FIG. 8 is a graph showing an influence of polyarginyl histidine on the growth of Fusobacterium nucleatum ATCC 25586 strain.

FIG. 9 is a graph showing an influence of polyarginyl histidine on the growth of Treponema denticola ATCC 33520 strain.

FIG. 10 is a graph showing an influence of polyarginyl histidine on the KGP activity of Porphyromonas gingivalis ATCC 33277 strain (Average±SD, n=3).

FIG. 11 is a graph showing an influence of polyarginyl histidine on the RGP activity of Porphyromonas gingivalis ATCC 33277 strain (Average±SD, n=3).

FIG. 12 is a graph showing influences of polylysine and polyarginyl histidine on the adhesion of Porphyromonas gingivalis ATCC 33277 strain to the saliva-coated hydroxyapatite beads (Average±SD, n=5).

FIG. 13 is a graph showing an influence of ployarginiy histidine on the coaggregation of Porphyromonas gingivalis ATCC 33277 strain and Streptococcus oralis ATCC 9811 strain.

### DETAILED DESCRIPTION OF THE INVENTION

Reference will now be made in detail to the preferred embodiments of the invention. This invention may, however, be embodied in many different forms and should not be construed as limited to the embodiments set forth herein. In addition, and as will be appreciated by one of skill in the art, the invention may be embodied as a method, system or process.

A composition for oral cavity of the invention contains a peptide as described above. The peptide is a peptide in which arginine and histidine bind alternately and is represented by the following general Formula (2) or (3). A preferable structure is the one represented by the following general Formula (2), that is, the one in which arginine and histidine link alternately with arginine being N-terminal. The composition for oral cavity of the invention may contain one or more kinds of the peptide in combination.

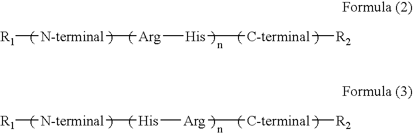

In the above general Formulae (2) and (3), “Arg” represents arginine and “His” represents histidine. In addition, R₁ represents hydrogen, sugar, acyl, biotinyl, thiol, phenol, or indole; R₂ represents a hydroxyl group, sugar, acyl, biotinyl, thiol, phenol, or indole; and n indicates an integer of 2 or more.

The peptide represented by the above general Formulae (2) and (3) is generally called polyarginyl histidine. This is applied to this description. Further, polyarginyl histidine can be abbreviated as “pRH.” “p” shows the abbreviation for poly, “R” shows a single character code of arginine, and “H” shows a single character code of histidine.

The arginine may be either D-arginine or L-arginine. In the general Formula (2) or (3), a preferable ratio of D-arginine and L-arginine is preferably approximately 10:90 to approximately 0:100, or more preferably approximately 0:100. This ratio is based on the analysis of D-/L-arginine and D-/L-histidine to be described later.

The histidine may be either D-histidine or L-histidine. In the general Formula (2) or (3), a preferable ratio of D-histidine and L-histidine is preferably approximately 100:0 to approximately 70:30, or more preferably approximately 100:0. This ratio is based on the analysis of D-/L-arginine and D-/L-histidine to be described later.

The analysis of D-/L-arginine and D-/L-histidine each constituting the polyarginyl histidine, that is to say, optical purity tests of arginine and histidine are described below.

Polyarginyl histidine was hydrolyzed by heating at 100° C. for 20 hours in a 6N hydrochloric acid solution to prepare the mixture of arginine and histidine. The prepared mixture of arginine and histidine was analyzed by means of high performance liquid chromatography (HPLC) on which an optical separation column (Daicel Industries CROWNPAK CR (+), manufactured by Daicel Chemical Industries, Ltd., a mobile phase is perchloric acid of pH 1.5, a column temperature is 4° C.) is placed. Detection was performed by measuring an absorbance at 200 nm. In a control experiment, four kinds of amino acid standards composed of D-histidine, L-histidine, D-arginine, and L-arginine, and the standard obtained through hydrolysis by heating two kinds of chemical synthesized polyamino acids (N-terminal L-Arg-D-His-L-Arg-D-His-L-Arg-D-His-L-Arg-D-His-L-Arg-D-His C-terminal and N-terminal L-Arg-L-His-L-Arg-L-His-L-Arg-L-His-L-Arg-L-His-L-Arg-L-His C-terminal) at 100° C. for 20 hours in a 6N hydrochloric acid solution, are analyzed by means of HPLC under the same condition. As a result, D-/L-arginine and D-/L-histidine are analyzed and the ratio thereof is determined.

In the general Formulae (2) and (3), as mentioned above, R₁ at a N-terminal represents hydrogen, sugar, acyl, biotinyl, thiol, phenol, or indole and R₂ at a C-terminal represents a hydroxyl group, sugar, acyl, biotinyl, thiol, phenol, or indole. R₁ at a N-terminal is preferably hydrogen. R₂ at a C-terminal is preferably a hydroxyl group.

In the invention, the degree of polymerization n of polyarginyl histidine is not particularly limited. However, the degree of polymerization n is preferably approximately 1 to approximately 20, and more preferably approximately 5. The polyarginyl histidine having the degree of polymerization of approximately 5 may be particularly preferably used because the polyarginyl histidine can be produced at a low cost by microbial fermentation.

The degree of polymerization is measured as follows: The molecular weight of the polyarginyl histidine is measured according to a MALDI-TOF Mass method (Matrix Assisted Laser Desorption/Ionization-Time of Flight Mass spectrometry) by using a time-of-flight mass spectrometer. The molecular weight of H₂O (approximately 18) is subtracted from the obtained molecular weight, and then the resultant is divided by sum of both the amounts of arginine residue (156.18) and histidine residue (137.14), thereby calculating the degree of polymerization. The amount of residue is a value obtained by subtracting the molecular weight of H₂O from amino acid molecule.

The sequence of polyarginyl histidine is analyzed by means of Edman degradation analyzer, (Model 492, manufactured by Applied Biosystems, Co., Ltd.). The analysis reveals that polyarginyl histidine contained in the composition for oral cavity of the invention has a structure in which arginine and histidine link alternately.

The polyarginyl histidine represented by the general Formula (2) or (3) is produced by a method known in the art such as a chemical synthesis method, a biochemical method, and microbial fermentation. For a method of producing the polyarginyl histidine which is contained in the composition for oral cavity of the invention at a low cost, a microbial fermentative production is most preferable.

Examples of the chemical synthesis methods of polyarginyl histidine include: the method that a C-terminal carboxyl group of a peptide binds covalently to a solid phase base and amino acids bind sequentially in an N-terminal direction, thereby a peptide synthesized, this method had being developed by R. B. Merrifield in 1963; the method of coupling of an α-NH₂-protected amino acid or an N-terminal-protected peptide, an α-carboxy-protected amino acid or a C-terminal-protected peptide with the protected side chain by using a condensing agent such as carbodiimide; the method of producing a peptide bond by using a reverse reaction of protease; and the method of polymerizing a side chain-protected arginine and N-carboxy anhydrides of side chain-protected histidine.

An example of the biochemical production method of polyarginyl histidine includes the method of producing polyarginyl histidine by means of genetic engineering in an intracelluar or acellular proteosynthetic system, by using DNA or RNA which codes the polyarginyl histidine, on the basis of functions regarding transcription and translation based which are inherent in organisms.

An example of the production method of polyarginyl histidine by microbial fermentation includes the method described in WO2004/014944 A1. The example of production method by microbial fermentation is described below.

In the production of polyarginyl histidine to be used for the invention, strains belonging to the genus Epichloe may be preferably used. Preferable microbes are Epichloe kibiensis E18 strains (FERM P-18923) or those variants. Epichloe kibiensis E18 strains (FERM P-18923) (hereinafter, referred to as “E18 strain”) are deposited with International Patent Organism Depositary, National Institute of Advanced Industrial Science and Technology, Central-6, 1-1, Higashi-1-chome, Tsukubashi, Ibaragi prefecture (Microbe Indication: Epichoo kibionsis E18). Derivative strains having improved higher productivity of polyarginyl histidine can be obtained by means of mutant induction, recombinant gene techniques using E18 strain as parental strain. Derivative strains include the strain in which mutation is artificially induced or the strain obtained through screening and so on.

The medium for the microbe producing polyarginyl histidine, which is contained composition of the invention, for example, for E18 strain and the like, may be arbitrarily selected depending on the characters of microbes and is available from commercial products, but can be prepared by methods known to one skilled in the art. Complete medium, synthetic medium, and semisythetic medium, each of which is comprised by an adequate composition in a liquid or solid form, can be used. However, the liquid medium is suitable in view of easiness of operation or the like. Any kinds of medium may be used as long as the medium contains, as a general ingredient, carbon source, nitrogen source, minerals, and other nutrients. Examples of carbon source include glucose, galactose, fructose, glycerol, and starch, and the content thereof is preferably approximately 0.1 to approximately 10% (w/v). Example of nitrogen source include organic compounds such as yeast extract, peptone, a casein hydrolysate, and amino acid; inorganic ammonium salts such as ammonium sulfate, ammonium chloride, and sodium nitrate, and the content thereof is preferably approximately 0.1 to approximately 5% (w/v). Other nutrients which provide minerals include a phosphate ion, a potassium ion, a sodium ion, a magnesium ion, a zinc ion, an iron ion, a manganese ion, a nickel ion, a sulfate ion, or the like; vitamins such as vitamin B₁; an antibiotic such as ampicillin, tetracycline, and kanamycin may be added to the medium as required.

Cultivation can be performed through shaking culture, stirred culture, or the like under aerobic conditions. A cultivation temperature is in a range of approximately 25 to approximately 40° C. A pH of the medium is approximately 2.0 to approximately 8.0, preferably approximately 3.0 to approximately 8.0, and more preferably approximately 5.0. A cultivation period is normally approximately 1 day to approximately 14 days, but the cultivation can be continued for more than approximately 14 days.

The above derivative strain (variant) derived from E18 strain as parental strain also can be cultivated in the same way.

A crude product is isolated from a culture by means of filtration or centrifugation when the produced polyarginyl histidine is secreted into the culture solution. The purification of the produced polyarginyl histidine can be performed by a method known in the art such as an ion-exchange resin treatment method, an activated carbon adsorption treatment method, an organic solvent precipitation method, a vacuum concentration method, a freeze dehydration method, and a crystallization method each of which is used for purification and isolation of natural or biosynthetic amino acids and proteins from recovered culture supernatant in appropriate combination. When the produced polyarginyl histidine presents in periplasm and cytoplasm of cultured microbe, the cells are collected by filtration or centrifugation, and a cell wall and/or a cell membrane thereof are destroyed by means of sonication and/or a lysozyme treatment, to finally obtain debris (cell fragments). The debris can be dissolved in an appropriate aqueous solution such as buffer, to thereby isolate and purify the product according to the above method.

The physical and chemical properties of polyarginyl histidine produced by E18 strain are described below:

(1) Only arginine and histidine are produced through hydrolysis with a 6N hydrochloric acid solution.

(2) Polyarginyl histidine and its hydrolysate indicate positivity for Sakaguchi reaction and Pauli reaction.

(3) A binding pattern between monomers is a peptide bond between α-carboxyl group and α-amino group.

(4) Amino acid sequence determined by an automated Edman degradation method presents alternative repetition between arginine and histidine, with N-terminal being arginine.

(5) In molecular weight measurement by MALDI-TOF Mass method (Matrix Assisted Laser Desorption/Ionization-Time of Flight Mass spectrometry), a molecule having molecular weight of about 1,486 is a main component. In addition to the molecule, there is a mixture of a different molecule with a difference in regular molecular weight of about 293.

(6) The produced histidine indicates, in thin-layer chromatography, an Rf value (0.19) that is the same as that of a histidine standard, and indicates positivity for ninhydrine reaction and Pauli reaction.

(7) The optical purity of the produced histidine is analyzed by means of chromatography spectrometry with an optical separation column. As a result, D type accounts for about 85% of the produced histidine.

The functional group released from polyarginyl histidine residue obtained by a chemical synthesis method, biochemical method, microbial fermentation, or the like can be subjected to various chemical modifications such as acylation. Those methods for derivatization are well known in the art. For example, a guanidino group released as a side chain from an arginine residue in polyarginyl histidine can be alkaline hydrolyzed, and is changed to ornithine. In arginine and ornithine, an acid dissociation constant of each guanidino group and amino group is different, a composition ratio of arginine and ornithine can be appropriately adjusted, to thereby produce polyarginyl histidine having the acid dissociation constant (pKa) of an electrolytic functional group optimal for application purposes.

Polyarginyl histidine contained in the composition for oral cavity of the invention does not show, as will be described later in experimental examples, a remarkable growth inhibitory activity for oral microbe. Furthermore, the polyarginyl histidine does not inhibit enzyme activities of an arginine-specific protease and lysin-specific protease as dominant virulence factor for Porphyromonas gingivalis.

On the contrary, as described later in experimental examples, polyarginyl histidine contained in the composition for oral cavity of the invention suppresses a hemagglutination activity of Porphyromonas gingivalis as an oral microbe. Furthermore, the polyarginyl histidine inhibits the adhesion of Porphyromonas gingivalis and saliva-coated hydroxyapatite. Furthermore, the polyarginyl histidine suppresses the coaggregation of Porphyromonas gingivalis and Streptococcus oralis.

The composition for oral cavity of the invention contains polyarginyl histidine which suppresses a hemagglutination activity of Porphyromonas gingivalis, the adhesion of Porphyromonas gingivalis and saliva-coated hydroxyapatite, and the coaggregation between Porphyromonas gingivalis and Streptococcus oralis, so that the composition can prevent periodontopathic bacteria from adhering to oral tissue. This effect according to the invention is effective for prevention and treatment of periodontal diseases such as periodontitis, gingivitis, pericementitis, pericoronitis of wisdom tooth, pericoronitis of implant, and various symptoms and diseases which are dental caries, stomatitis, halitosis, and the like. The composition of the invention can be also used for the periodontal diseases, various symptoms and diseases. Of those, the composition of the invention is suitable for prevention and treatment of periodontal diseases. Therefore, the composition of the invention can be administered for preventing and treating the periodontal diseases.

According to a general method, the composition for oral cavity of the invention is produced by formulating polyarginyl histidine. According to a formulation form, the composition for oral cavity of the invention is produced by a method in common use such as mixture, kneading, granulation, making tablet, coating, sterilization, emulsification, or the like. A blending amount of polyarginyl histidine in the composition for oral cavity is approximately 0.001 to approximately 10 wt %, or preferably approximately 0.01 to approximately 1 wt %.

The form of the composition for oral cavity of the invention is not particularly limited as long as it is the composition for oral cavity and may be administered orally. The composition can be formulated as, for example, a solid medicine such as a tablet, ball, granule, subtle granules, powder, capsule, troche, chewable, and gum; liquid medicine such as emulsion, suspension, syrup, and elixir; gels; and ointment. Those formulation methods can be produced by a known method. Furthermore, on the formulation, an appropriate carrier or the like can be selected in accordance with the form, to thereby be formulated.

The composition for oral cavity of the invention can be appropriately formulated with optional components to the extent that an effect of polyarginyl histidine is not impaired. Examples of such optional component include: an abrasive, coking agent, thickener, wetting agent, sweetening, flavoring substance, correctives, fragrance, antiseptic agent, pH adjuster, pigment, diluting agent, binding agent, lubricant agent, disintegrant, emulsifying agent, nonaqueous vehicle, antioxidizing agent, tonic solution, suspending agent, preservative, solubilizing agent, dispersing agent, thickening agent, plasticizing agent, absorbing agent, antioxidant agent, and other agents.

The composition for oral cavity of the invention can take various forms such as, for example, a dentifrice such as a paste dentifrice, powder dentifrice, liquid dentifrice, and frothy dentifrice; gingival massage cream; local embrocation; mouthwash; gargle; mouth freshener; and chewing gum. Of those forms, preferable form includes a paste dentifrice, gargle, mouthwash, chewing gum, and the like, and more preferable form includes a paste dentifrice, mouthwash, and chewing gum.

### EXAMPLES

The following examples are for illustrative purposes only and are not intended, nor should they be interpreted to, limit the scope of the invention. In particular, an experimental example demonstrating effects of polyarginyl histidine will be described.

Sample: Production Example of Ployariginyl Histidine (pRH)

Initial Binding of Amino Acid to Resin

6 g of Fmoc-D-His(Trt)-OH (Cas No. 135610-90-1: manufactured by Merck Ltd.) and 5 g of diisopropylethylamine (Cas No. 7087-68-5) (hereinafter, referred to as “DIPEA”) were dissolved in 20 mL of N,N-dimethylformamide (Cas No. 68-12-2) (hereinafter, referred to as “DMF”) and 50 mL of dichloromethane (Cas No. 75-09-2) (hereinafter, referred to as “DCM”). The dissolved solution was added with 5 g of 2-chlorotrityl chloride resin (manufactured by Merck Ltd.) (hereinafter, referred as “2-ClTrt resin”), mixed at 30° C. for 2 hours, and subjected to reaction.

After the reaction, the obtained resin was washed with about 50 mL of a solvent which will be described later. The washing was carried out sequentially three times by DCM:MeOH:DIPEA (17:2:1), three times by DCM, two times by DMF, and two times by DCM. After that, the obtained resin was dried in a vacuum drying on KOH, and a Fmoc-D-His(Trt)-resin was obtained.

Deprotective Operation of Protective Fmoc Group of α-Amino Group

A whole amount of the synthesized Fmoc-D-His(Trt)-resin (or a Fmoc protective peptidyl resin) was added to about 50 mL of a DMF solution containing piperidine (Cas No. 110-89-4) of 20% (v/v), shaked at 30° C. for 3 hours, and the solution was discarded. The same processing was repeated three times to four times, and the resin was finally washed with about 50 mL of DMF.

Coupling Operation

26 g of Fmoc-L-Arg(pbf)-OH (Cas No. 154445-77-9: manufactured by Merck Ltd.), 14.9 g of HBTU (Cas No. 94790-37-1), and 5.4 g of 1-hydroxybenzotriazole (Cas No. 2592-95-2) (hereinafter, referred to as “HOBt”) were dissolved in about 80 mL of DMF, and mixed after addition of 10 g of DIPEA. The mixture was immediately added to the resin (N-terminal presents H is (Trt)) that had been subjected to the deprotective operation of the protective Fmoc group of α-amino group, and subjected to reaction at 30° C. for 2 hours.

When the resin (N-terminal presents Arg (pbf) that had been subjected to the deprotective operation of the protective Fmoc group of the α-amino group was bound with histidine, the same coupling operation was performed by adjusting Fmoc-D-His(Trt)-OH to 25 g.

Elongation for Peptide Chain

The deprotective operation and the coupling operation of the protective Fmoc group of the α-amino group were repeated until a peptide of target chain length was obtained.

In the present experiment, arginine and histidine were alternately bonded, and the deprotective operation and the coupling operation of the protective Fmoc group of α-amino group were repeated until a pentamer of (arginine-histidine) was formed.

Peptide Excision from Resin

First, a deprotective operation of protective Fmoc group of α-amino group of resin was performed to remove the Fmoc group at N-terminal. Next, the resin was washed five times each with about 50 mL of DMF-acetic acid (60:40) and 50 mL of DCM. Finally, the resin was washed 5 times with methanol of about 50 mL, and was dried in a vacuum on KOH overnight.

25 mL of a mixture comprising Trifluoroacetic acid (hereinafter, referred to as “TFA”) (Cas No. 76-05-1), Triisopropylsilane (hereinafter, referred to as “TIS”) (Cas No. 6485-79-6) and water in respective ratios of 95:2.5:2.5 (volume ratio) was added to this resin, and left at room temperature for 24 hours with occasional agitation. The obtained solution was passed through a suction filtration, and the filtrate was collected. The resin was washed two times with 50 mL of the same mixture (TFA:TIS:water=95:2.5:2.5) and the filtrate was retrieved as well. Ice-cooled ethyl ether (2 L) was added dropwise to the retrieved solution to form a precipitate. The solution was passed through a suction filtration to be retrieved, and was further washed with a small amount of cooled ethyl ether. This precipitate was dried in a vacuum and a crude peptide was obtained.

Purification

The crude peptide was dissolved in 1% CH₃CN (0.1% TFA) solution at a concentration of 25 mg/mL, and purified by the following semi preparative column as follows:

| | |
|---|---|
| Sample | 4 mL (25 mg/mL) |
| Column | YMC Pack ODS-A, 20 mm I.D. × 250 mm |
| Eluent | 0.1% TFA, gradient CH₃CN 1% → 60% (80 minutes) |
| Flow Rate: | 5 mL/minute |
| Temperature: | Room Temperature |
| Detection | 220 nm |

The eluted part of HPLC was passed through a column with 30 mL of DOWEX cation exchange resin (H-type), washed with water, and was eluted with 1 mol/L of hydrochloric acid. The eluted part was lyophilized and about 48 g of pRH was obtained.

Physical Properties of Polyarginyl Histidine

Purity Test

Polyarginyl histidine was analyzed with the high performance liquid chromatography (HPLC). A sample of 0.5 μl of polyarginyl histidine (0.01 mg/μl) was measured in the following condition. In this case, the peak purity was 98.5%.

| | |
|---|---|
| Column | YMC Pack ODS-A, 4.6 mm I.D. × 150 mm |
| Eluent | 0.1% TFA, gradient CH₃CN 1% → 60% (25 minutes) |
| Flow Rate: | 1 mL/minute |
| Temperature: | Room Temperature |
| Detection | 220 nm |

Amino-Acid Analysis

After polyarginyl histidine was hydrolyzed with 6N HCl at 110° C. for 22 hours, quantity of amino acid was determined by means of an amino acid analyzer (L-8800 manufactured by Hitachi). As a result, a molar ratio [arginine:hisdinie=5.00:5.01] was obtained.

Furthermore, an elemental analysis of ployarginyl histidine was carried out. The result of the elemental analysis is shown in table 1. Theoretical values and experimental values were substantially corresponding.

TABLE 1

| | C % | H % | N % |
|---|---|---|---|
| Experimental Value | 36.07 | 6.08 | 24.34 |
| Theoretical Value* | 36.05 | 6.08 | 24.52 |

Theoretical value* Calculated value from C₆₀H₉₇N₃₅O₁₁•11HCl•6.3H₂O

Mass Spectrometry

A mass spectrographic measurement was performed based on Deconvolution method. Ionization was carried out by means of Electrospray Ionization Mass Spectrometry (ESI-MS). The measurement result was 1485 which was substantially corresponding with the theoretical value of 1484.64.

### Experimental Example 1

### Antimicrobial Effect on Periodontopathic Bacteria of Polyarginyl Histidine

Microbe Strains:

| | |
|---|---|
| 1. | Porphyromonas gingivalis ATCC 33277 strain, W50 strain |
| 2. | Actinobacillus actinomycetemcomitans ATCC 29523 stain, Y4 strain |
| 3. | Prevotella intermedia ATCC 49046 strain |
| 4. | Prevotella nigrescens ATCC 25261 strain |
| 5. | Fusobacterium nucleatum ATCC 23726 strain, ATCC 25586 strain |
| 6. | Treponema denticola ATCC 33520 strain |

Experimental Content 1

Each of the above cryopreserved strains was anaerobically cultivated at 35° C. for 48 hours by using TSB medium to which yeast extract (1 g/L), hemin (5 mg/mL), and menadione (1 mg/L) were added to provide a precultured bacterial culture.

100 μl of precultured bacterial culture were added to 5 mL of TBS medium containing each of pRH solution (1 μg/mL, 5 μg/mL, 10 μg/mL, 50 μg/mL, 100 μg/mL, 500 μg/mL), and optical densities (O.D._{660 nm}) were measured after 24 hours and 48 hours, respectively. The value obtained by subtracting the O.D._{660 nm} value in a case where only a pRH solution was added to the medium from the optical density was defined as turbidity by bacteria.

Experimental Result 1

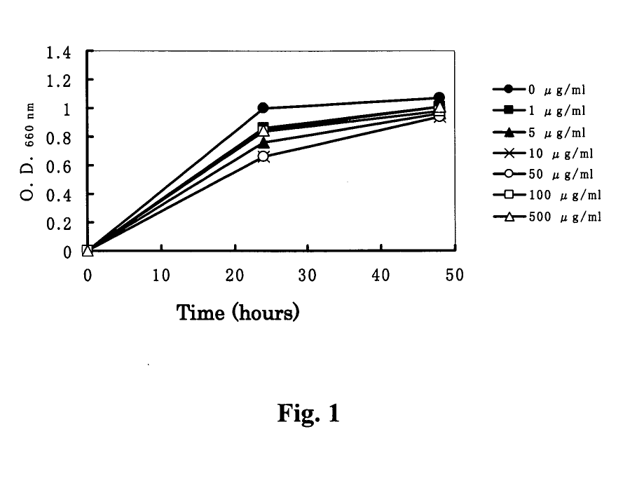

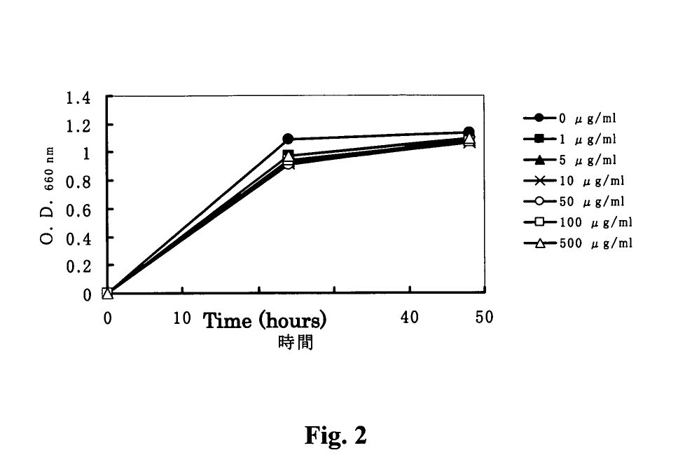

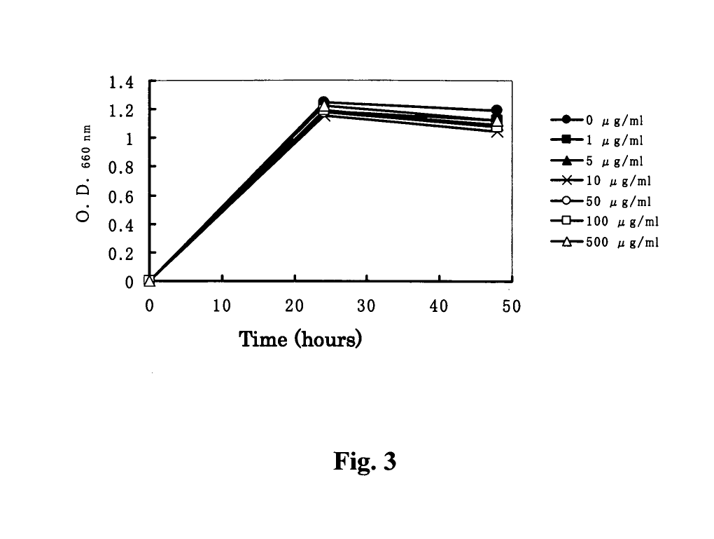

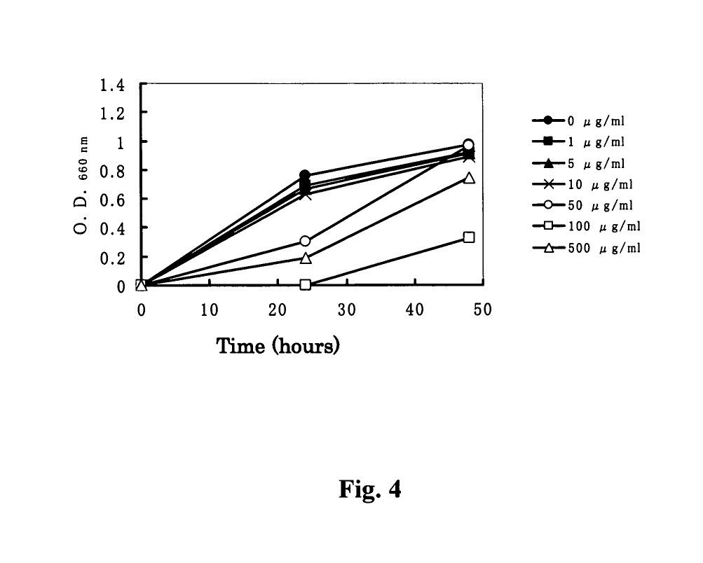

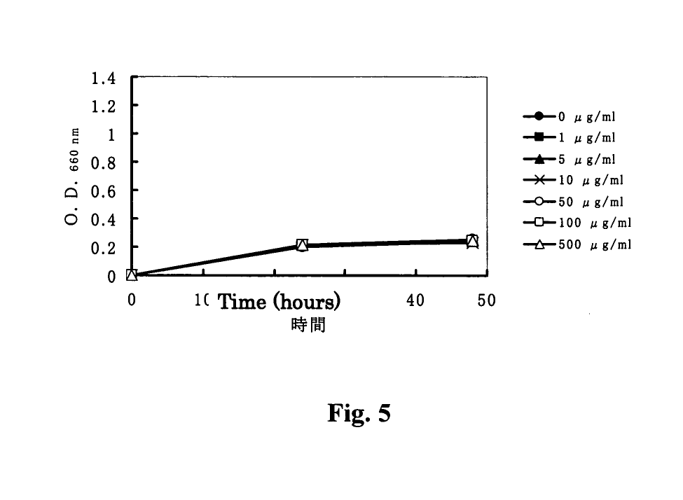

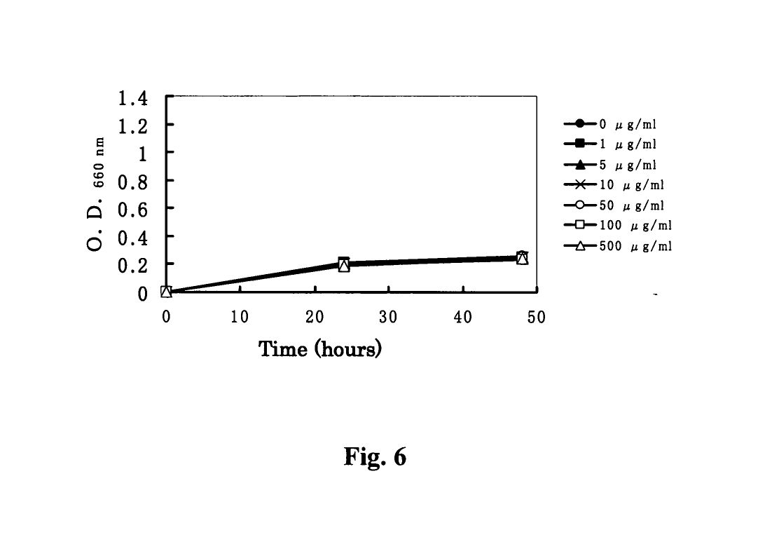

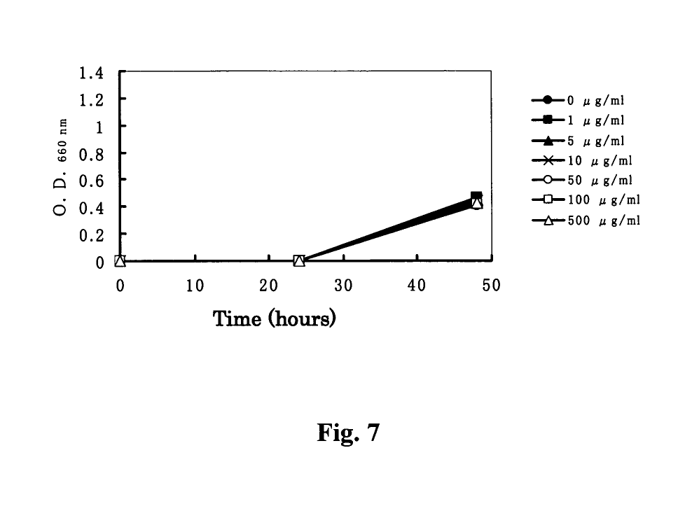

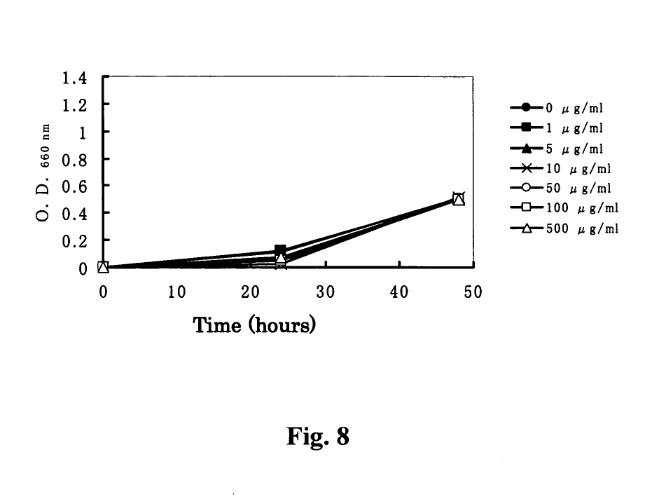

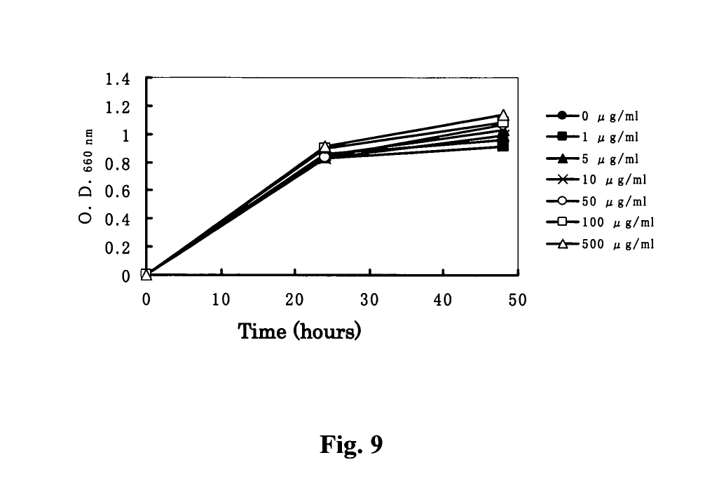

A growth inhibitory effect with the concentration of 100 μg/mL or more was observed in the pRH with respect to Prevotella nigrescens ATCC 25261 strain (FIG. 4), but the growth inhibitory effect with the concentration of 500 μg/mL was not observed with respect to other test strains (FIGS. 1 to 3, FIGS. 5 to 9). As described above, polyarginyl histidine did not show a significant growth inhibition activity for oral microbes.

### Experimental Example 2

### Influence of Polyarginyl Histidine on Protease of Porphyromonas gingivalis

Enzyme activity inhibitions of an arginine-specific protease (Arg-gingipain; hereinafter, referred to as “RGP”) and lysin-specific protease (Lys-gingipain; hereinafter, referred to as “KGP”) as dominant virulence factor for Porphyromonas gingivalis were examined.

Experimental Content 2

pRH (final concentrations of 100 μg/mL and 1,000 μg/mL) and a substrate (Bz-Arg-methylcoumarinamide for RGP, and Boc-Val-Leu-Lys-methylcoumarinamide for KGP: final concentrations of 100 μM respectively) were dissolved in 800 μl of Tris-HCl buffer (pH 7.6) added with NaCl (100 mM), CaCl₂ (5 mM), and cystein (10 mM), 200 μl of a culture supernatant of Porphyromonas gingivalis, and reacted at room temperature for 15 minutes. The reaction was stopped by adding 2 mM TLCK, and released methyl-coumarin-amide was measured by means of spectrophotofluorometer (excitation wavelength: 380 nm, fluorescence wavelength: 460 nm).

Furthermore, an inhibition rate was defined according to the Equation (1):

Inhibition rate(%)=(Blank fluorescence intensity−Sample fluorescence intensity)÷Blank fluorescence intensity×100 (1)

Blank fluorescence intensity indicates fluorescence intensity upon reaction without adding pRH. Sample fluorescence intensity indicates fluorescence intensity upon reaction with adding pRH.

Experimental Result 2

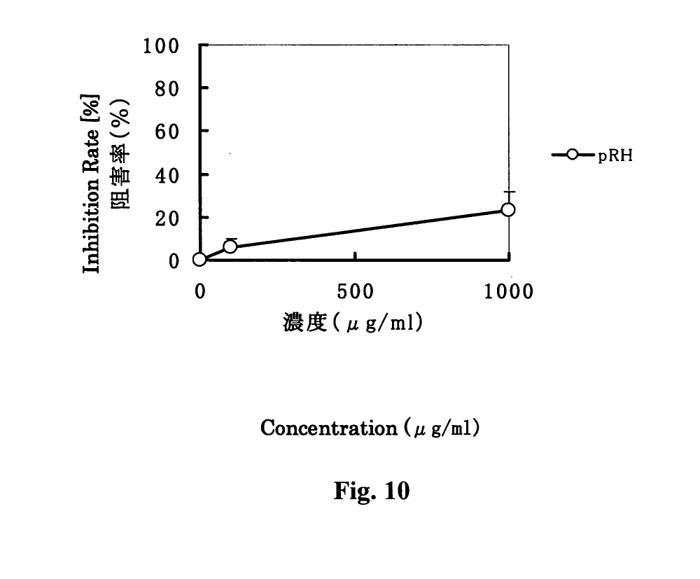

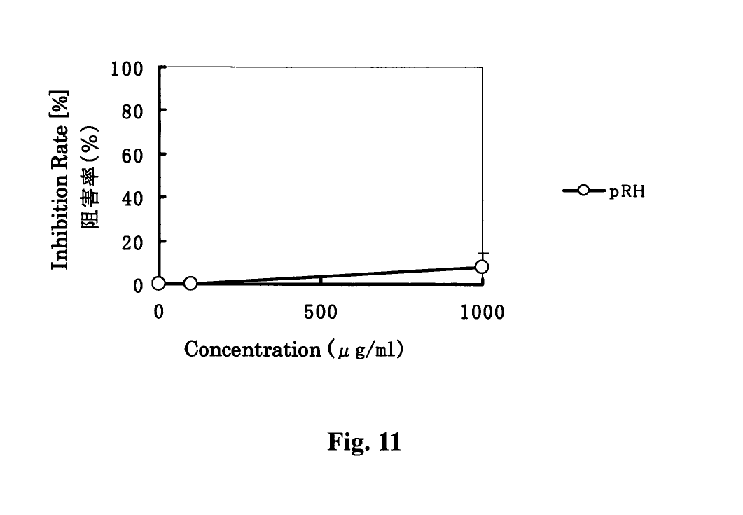

pRH at 100 μg/mL showed an inhibition rate of 5.7% for KGP, and pRH at 1000 μg/mL showed an inhibition rate of 23% for KGP (FIG. 10). In the case of RGP, pRH at 100 μg/mL showed an inhibition rate of 0.2%, and pRH at 1000 μg/mL showed an inhibition rate of 7.7% (FIG. 11). pRH did not indicate any remarkable inhibitions for KGP and RGP.

### Experimental Example 3

### Influence of Polyarginyl Histidine on Hemagglutination Activity of Porphyromonas gingivalis

Porphyromonas gingivalis has strong hemagglutination activity, so an inhibitory effect on hemagglutination activity was examined.

Experimental Content 3

A culture supernatant of Porphyromonas gingivalis with continuous 2-fold dilution, 90 μl of Porphyromonas gingivalis fungus bodies adjusted to O.D.₆₆₀ₙₘ=2, and 20 μl of sample polyarginyl histidine (final concentrations of 10 μg/mL, 100 μg/mL, and 500 μg/mL) were added into a 96 well microtiter plate, and 90 μl of a test solution obtained by suspending 1 mL of blood collected from human in 49 mL of physiological saline was added. After standing at room temperature for 2 hours, a minimum concentration at which a hemagglutination activity can be observed through naked-eye was determined.

Experimental Result 3

TABLE 2

| | Control | Polyarginyl Histidine | | |
|---|---|---|---|---|
| | 0 | 10 | 100 | 500 |
| | μg/mL | μg/mL | μg/mL | μg/mL |
| Porphyromonas gingivalis | 2⁷ | 2⁴ | 2² | 2¹ |
| Fungus Body (O.D. = 2) | | | | |
| Culture Supernatant of | 2⁹ | 2⁷ | 2⁶ | 2⁶ |
| Porphyromonas gingivalis | | | | |

Table 2 shows that a hemagglutination activity of Porphyromonas gingivalis decreased with increasing concentration of polyarginyl histidine. It was found that pRH inhibited the hemagglutination activity of Porphyromonas gingivalis depending on concentration of polyarginyl histidine.

### Experimental Example 4

### Influence of Polyarginyl Histidine on Adhesion of Porphyromonas gingivalis to Saliva-Coated Hydroxyapatite Beads

The fimbriae of Prophyromonas gingivalis has reported to specifically bind to proline-rich protein, proline-rich glycoprotein, and statherin of salivary proteins. Accordingly, the influence of polylysine and arginyl histidine on the adhesion on Porphyromonas gingivalis to saliva-coated hydroxyapatite beads was examined.

Experimental Content 4

2 mg of hydroxyapatite beads (hereinafter, referred to as “HA”) were incubated with 150 μl of nonstimulated human saliva at room temperature overnight, and washed with KCl buffer (50 mM KCl, 1 mM KH₂PO₄, 1 mM CaCl₂, 0.1 M MgCl₂). The obtained saliva-coated hydroxyapatite beads were hereinafter referred to as “sHA.” This sHA was added with Porphyromonas gingivalis (2×10⁸ cells) radiolabeled through cultivation on the medium with ³H of 5 μCi/mL, and each pRH or polylysine (0.1 mg/mL, 1 mg/mL, 10 mg/mL), incubated at room temperature for 1 hour with gently agitating, and washed with percoll and KCl buffer, to thereby obtain a sample. In addition, sHA was added with only radiolabled Porphyromonas gingivalis (2×10⁸ cells), incubated at room temperature for one hour with gently agitating, to thereby obtain washed with and percoll and KCl buffer, to thereby obtain a blank sample.

The ³H of Porphyromonas gingivalis binding to the sHA of those samples were measured, and an inhibition rate was calculated according to the following Equation (2):

Inhibition rate(%)=(Blank ³H value−Sample ³H value)÷Blank ³H value×100 (2)

A blank ³H value means a ³H measurement of Porphyromonas gingivalis in a blank sample treated without adding pRH or polylysine. A sample ³H value means a ³H measurement of Porphyromonas gingivalis in a sample treated with adding pRH or polylysine.

Experimental Result 4

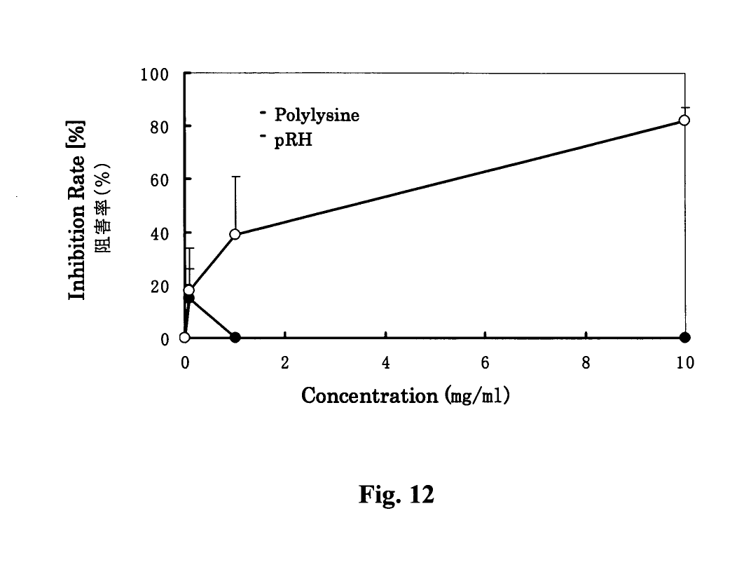

pRH obviously inhibited the absorption of Porphyromonas gingivalis for sHA depending on the concentration of pRH. On the other hand, polylysine, which was a basic peptide as well as pRH, did not inhibit the absorption of Porphyromonas gingivalis for sHA (FIG. 12).

### Experimental Example 5

### Influence of Polyarginyl Histidine on Adhesion Porphyromonas gingivalis to Early Dental Biofilm-Forming Bacterium

For colonization of Porphyromonas gingivalis, the adhesion to oral indigenous Gram-positive bacterium (coaggregation), which has been already fixed on tooth surface, is thought to be essential. Thus, the coaggregation of Porphyromonas gingivalis and Streptococcus oralis, which is one of the representative early dental biofilm-forming bacterium, was measured by a turbidity measurement method.

Experimental Content 5

Measurement of Coaggregation Activity

Porphyromonas gingivalis and Streptococcus oralis (each 5×10⁸ cells/mL) were added into 10 mM Phosphate buffered saline (PBS) (pH 6.0) as reaction solution, and a change in O.D. 550 nm was continuously recorded for 7.5 minutes by spectrophotometer (UV-265W; manufactured by Shimadzu Co., Ltd.) with agitating at 37° C. The continuous record for 7.5 minutes means the record continuously and automatically calculated an absorbance difference in 0.5 minute before and after some point, (i.e., an absorbance difference in 1 minute each). A maximum amount of absorbance change from obtained measurement values was read, and defined as A. Furthermore, Porphyromonas gingivalis (5×10⁸ cells/mL) were only added into 10 mM PBS (pH 6.0) as reaction solution, and a maximum amount of absorbance change was defined as B on the same measurement. Accordingly, a coaggregation activity is calculated from the following Equation (3):

Coaggregation Activity=A−B (3)

Measurement of Inhibition Rate

A pRH solution (final concentrations of 0.1 mg/mL, 0.5 mg/mL, 1 mg/mL, and 2.5 mg/mL) was added upon the measurement of a coaggregation activity, and a change in O.D. 550 nm was continuously recorded for 7.5 minutes, and a maximum amount of absorbance change was read as described above. In calculating of an inhibition rate, the coaggregation activity without adding a pRH solution defined as C, and the coaggregation activity with adding a pRH solution defined as D. The inhibition rate is calculated from the following Equation (4):

Inhibition Rate(%)=(C−D)÷C×100 (4)

Experimental Result 5

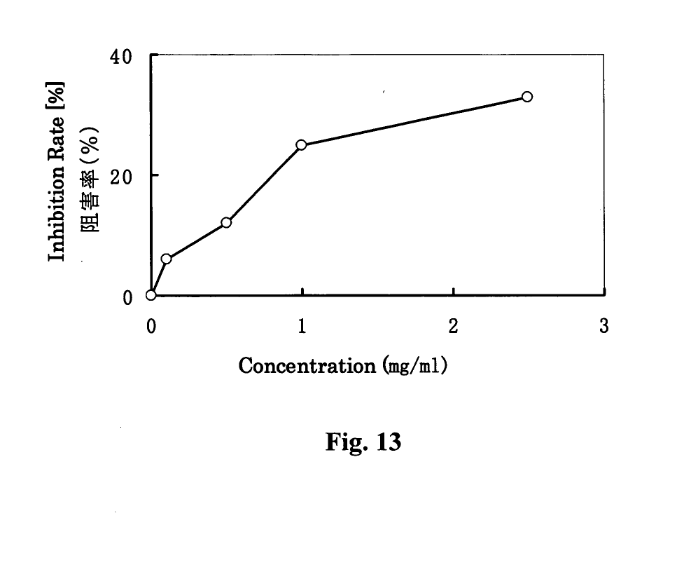

A pRH solution at the concentration of 2.5 mg/mL indicated an aggregation inhibitory activity of 33% (FIG. 13).

When only Streptococcus oralis was added, the maximum amount of absorbance change measured in a similar way as that of the maximum amount of absorbance change (A) when both Porphyromonas gingivalis and Streptococcus oralis were added indicated 1% or less of A. On the other hand, when only Porphyromonas gingivalis was added, the maximum amount of absorbance change (B) measured in a similar way indicated about 30% of A.

1. A method for treating an oral cavity comprising providing to a patient in need thereof with an oral composition comprising a peptide with alternately binding arginine and histidine residues within the structure of said peptide;
wherein said peptide inhibits at least one of (i) the hemagglutination activity of Porphyromonas gingivalis, (ii) the adhesion between Porphyromonas gingivalis and saliva coated hydroxyapatite beads and (iii) a congregation of Porphyromonas gingivalis and Streptococcus oralis.

2. The method of claim 1, wherein the peptide has a structure represented by at least one of Formula (I) and (II):
R₁-(Arg-His)ₙ-R₂ Formula (I)
R₁-(His-Arg)ₙ-R₂ Formula (II)
wherein, Arg represents arginine, and H is represents histidine; R₁ represents hydrogen, sugar, acyl, biotinyl; R₂ represents a hydroxyl group, sugar, acyl, biotinyl, thiol, phenol, or indole; and n represents an integer of 2 or more

3. The method of claim 1, wherein the peptide has a structure represented by the following Formula (1):
R₁-(Arg-His)ₙ-R₂ Formula (I)
wherein, Arg represents arginine, and H is represents histidine; R₁ represents hydrogen, sugar, acyl, biotinyl; R₂ represents a hydroxyl group, sugar, acyl, biotinyl, thiol, phenol, or indole; and n represents an integer of 2 or more.

4. The method of claim 1, wherein the oral composition further comprises at least one agent suitable for combining with said peptide to formulate said oral composition

5. The method of claim 1, wherein the ratio of D-arginine and L-arginine in the arginine is approximately 10:90 to approximately 0:100.

6. The method claim 1, wherein the arginine is L-arginine.

7. The method of claim 1, wherein the ratio of D-histidine and L-histidine in the histidine is approximately 100:0 to approximately 70:30.

8. The method of claim 1, wherein the histidine is D-histidine.

9. The method of claim 1, wherein the peptide is any one of a monomer to an icosamer of (arginine-histidine).

10. The method of claim 1, wherein the peptide is a pentamer of (arginine-histidine).

11. The method of claim 1, wherein the peptide is a peptide produced by microbial fermentation.

12. The method of claim 1, wherein the content of the peptide is approximately 0.001 to approximately 10 wt %.

13. The method of claim 1, wherein the content of the peptide is approximately 0.01 to approximately 1 wt %.

14. The method of claim 4, wherein said at least one agent suitable for combining with said peptide to formulate said oral composition is at least one of an abrasive agent, coking agent, thickener, wetting agent, sweetening agent, flavoring substance, correctives, fragrance, antiseptic agent, pH adjuster, pigment, diluting agent, binding agent, lubricant agent, disintegrant, emulsifying agent, nonaqueous vehicle, antioxidizing agent, tonic solution, suspending agent, preservative, solubilizing agent, dispersing agent, thickening agent, plasticizing agent, absorbing agent, and an antioxidant agent.

15. The method of claim 1, wherein said oral composition is formulated as a component of at least one of a paste dentifrice, a powder dentifrice, a liquid dentifrice, a frothy dentifrice, a gingival massage cream, a local embrocation, a mouthwash, a gargle, a mouth freshener, and a chewing gum.

16. The method of claim 1, wherein the concentration of the peptide in said oral composition is approximately 10 μg/mL to approximately 1,000 μg/mL.

17. The method of claim 15, wherein the concentration of the peptide oral composition is approximately 100 μg/mL to approximately 500 μg/mL.
